(19) United States
(12) Patent Application Publication
(10) Pub. No.: US 2003/0198657 A1
(43) Pub. Date: Oct. 23, 2003

(54) Methods using phytol to improve the appearance of skin and compositions for such methods

(75) Inventors: Gopinathan K. Menon, Wayne, NJ (US); Dmitri Ptchelintsev, Jersey City, NJ (US); Harish Mahalingam, Ledgewood, NJ (US)

Correspondence Address: CHARLES N.J. RUGGIERO, ESQ., OHLANDT, GREELEY, RUGGIERO & PERLE, L.L.P., 10th FLOOR, ONE LANDMARK SQUARE, STAMFORD, CT, 06901-2682, US

(73) Assignee: Avon Products, Inc.

(21) Appl. No.: 10/442,219

(22) Filed: May 20, 2003

Related U.S. Application Data
(63) Continuation of application No. 09/724,356, filed on Nov. 28, 2000, now abandoned.
(60) Provisional application No. 60/190,988, filed on Mar. 21, 2000.
(60) Provisional application No. 60/190,989, filed on Mar. 21, 2000.

(51) Int. Cl. A61K 31/203; A61K 7/00; A61K 31/07; A61K 31/045

(52) U.S. Cl. 424/401; 514/725; 514/559; 514/703; 514/729

(57) ABSTRACT

There are provided methods of enhancing the appearance of human skin comprising applying a composition having (i) phytol in an amount about 0.0001 wt % to about 50 wt % based on the total weight of the composition, and (ii) at least one retinoid in an amount about 0.001 wt % to about 1.5 wt % based on the total weight of the composition. Alternatively, the composition used in the method for enhancing the appearance of human skin may have phytol and perilla oil.

### RELATED APPLICATIONS

This application claims priority in U.S. applications Ser. No. 60/190,988, filed Mar. 21, 2000; Ser. No. 60/190,989, filed Mar. 21, 2000; and Ser. No. 09/521,442, filed Mar. 7, 2000.

### BACKGROUND OF THE INVENTION

1. Field of the Invention

The present invention relates to methods for improving the appearance of skin. In particular, the present invention relates to such methods in which topical compositions having phytol in combination with selected ingredients are applied to the skin.

2. Description of the Prior Art

Numerous methods have been developed that attempt to improve the appearance of human skin. Many of the more effective methods employ topically applied compositions containing one or more active ingredients known to beneficially affect the skin.

For example, there are methods that employ compositions containing retinoids, particularly retinol. Retinoids induce and/or promote the biosynthesis and/or bioactivity of endogenous chemicals when topically applied to the skin. Specifically, retinoids bind to retinoic acid receptors (RARs) and retinoid X receptors (RXRs) in the skin. Compositions having retinoid may be used to treat a myriad of unwanted skin conditions, such as acne and wrinkles. However, consumers with sensitive skin cannot tolerate high levels of retinoids in cosmetic compositions.

A composition having phytol is described in JP 10-114648 (1998) to Watanabe et al. The Watanabe application is directed to topical compositions for the prevention and treatment of acne. The effective ingredient of these topical compositions is at least one C₂₀ to C₂₅ terpene alcohol, such as phytol. This citation discloses that therapeutic medicines for common acne can have enhanced effects by using the terpene alcohols with other known anti-acne compounds, such as retinoids.

### SUMMARY OF THE INVENTION

It is an object of the present invention to provide a method for enhancing the appearance of human skin.

It is another object of the present invention to provide such a method that employs a composition having phytol and a retinoid, in which phytol is present in an amount that, along with the retinoid, is effective to synergistically enhance the skincare benefits of the retinoid.

It is yet another object of the present invention to provide such a method employing a composition having phytol and a retinoid, in which phytol is present in an amount effective to decrease the adverse effects of the retinoid on human skin.

It is still a further object of the present invention to provide a method for enhancing the appearance of human skin, namely reducing dermatological aging, particularly dermatological aging due to intrinsic and/or extrinsic aging, such as photo-aging and the effects of pollution; decreasing skin fragility; preventing and reversing loss of collagen; preventing skin atrophy; promoting/accelerating cell turnover; improving skin firmness/plumpness; improving skin texture; decreasing fine lines and wrinkles; improving skin tone; enhancing skin thickness; decreasing pore size; minimizing skin discoloration; restoring skin luster; minimizing signs of fatigue; and preventing, reducing or treating hyperpigmentation, especially associated with blemishes, improving skin barrier functions, minimizing skin dryness; and combinations of the foregoing.

It is still yet another object of the present invention to provide a method for enhancing the appearance of human skin and preventing, reducing or treating acne that comprises applying to the human skin a composition having phytol and perilla oil.

These foregoing objects, as well as other objects and advantages, are achieved by methods of enhancing the appearance of human skin comprising applying a composition having (i) phytol in an amount about 0.0001 percent by weight (wt %) to about 50 wt % based on the total weight of the composition, and (ii) at least one retinoid in an amount about 0.001 wt % to about 1.5 wt % based on the total weight of the composition.

In an alternative embodiment, the composition used in the method for enhancing the appearance of human skin may have phytol and perilla oil.

### DETAILED DESCRIPTION OF THE INVENTION

The present invention is directed to methods of improving the appearance of skin and compositions useful in such methods. Skin conditions that may be treated by methods described herein include dry skin, photodamaged skin, wrinkles, age spots, pigmentary disorders, skin lightening, psoriasis and atopic dermatitis.

Some methods according to the present invention may comprise the step of topically applying a composition having phytol and a retinoid. Alternatively, two separate compositions may be contemporaneously applied, in which one composition has phytol and the other composition has a retinoid.

Other methods according to the present invention may comprise the step of topically applying a composition having phytol and perilla oil.

Phytanic acid is metabolized from phytol when phytol is applied to the skin. Phytanic acid provides independent beneficial skincare effects. In addition, phytanic acid also provides a synergistic effect in combination with a retinoid, which provides optimal skincare effects by globally activating retinoic acid receptors (RARs), retinoid X receptors (RXRs), and peroxisome proliferator activated receptor (PPAR) responsive genes.

It has been discovered that phytol induces and promotes the biosynthesis and/or bioactivity of endogenous chemicals when topically applied to the skin. This discovery is described in commonly owned U.S. Provisional Patent Application Serial No. 60/190,988, which was filed on Mar. 21, 2000 and is incorporated herein by reference.

Significantly, it has now been unexpectedly discovered that phytanic acid activates or “upregulates” PPAR responsive genes. Upregulation of PPAR responsive genes has been documented to improve the skin barrier function and, hence, moisture retention by skin. As a result, both extrinsic and intrinsic skin aging are reduced by topically applying compositions containing phytanic acid precursors, such as phytol. In addition, adverse or deleterious effects of retinoids, such as dry skin, flaky skin and/or reddened skin (i.e. sensitized), are minimized by retention of moisture in the skin due to PPAR receptor upregulation.

Moreover, phytanic acid is a recognized RXR agonist competing with retinoids, such retinol, at the receptor site. Thus, phytanic acid will bind and activate available RXR sites, leaving only RAR sites available for binding and activating with a lesser or smaller level of retinoids. Thus, combining phytanic acid with one or more retinoids enhances the efficacy of the retinoids by increasing the number of RAR sites activated absent the presence of phytanic acid.

In light of the foregoing, it is believed that methods according to the present invention provide multiple skin care benefits by simultaneously and/or contemporaneously activating RXRs, PPARs, and, preferably, RARs. Activating these receptors and responsive genes stimulates cell functions in multiple components of skin, such as the epidermis, dermis, sebaceous glands, melanocytes, Langerhan cells, and hair follicles.

Phytol and its derivatives are hereby defined as organic compounds that generally comply with the following structural formula:

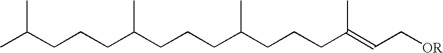

where R is selected from a group of substituents that includes hydrogen, as well as cyclic and acyclic hydrocarbon residues, which may contain one or several unsaturated bonds and/or heteroatomic substituents. The preferred substituents are hydrogen, acyls and cyclic or linear alkyls.

Furthermore, the term “phytol” as used herein encompasses phytol, phytol derivatives, phytol precursors, and phytol metabolites, preferably phytanic acid. Metabolic precursors of phytol are hereby defined as compounds from which phytol can be formed by action of enzymes present in human tissues, particularly skin.

The term “retinoid” includes: (1) retinol; (2) esters of retinol with carboxylic acids of 1 to 24 carbon atoms, such as retinyl acetate, retinyl propionate, retinyl butyrate, retinyl octanoate, retinyl laurate, retinyl palmitate, retinyl oleate, retinyl linoleate; (3) esters of retinol having an alpha-hydroxy carboxylic acid; (4) ether derivatives of retinol, including alkyl ether, ethers derived from glycolic acid, as well as glycolate ester and amide, such as retinyl glycolyl ether; (5) retinaldehyde; (6) retinoic acid; (7) esters of retinoic acid with alcohols of 1 to 24 carbon atoms; (8) isotretinoin as well as synthetic retinoid mimics, and derivatives of the foregoing, as well as others that bind to RAR receptors; (9) cis- and trans-isomers of the foregoing retinoids; (10) salts of the foregoing retinoids; and (11) mixtures of the any of the foregoing compounds. A preferred retinoid for use in a composition according to the present invention is retinol. A more preferred retinoid for use in a composition according to the present invention is the trans-isomer of retinol.

Compositions that can be used in the methods of the present invention will have phytol in an amount about 0.0001 percent by weight (wt %) to about 50 wt % based on the total weight of the composition. Preferably, phytol may be present in an amount about 0.01 wt % to about 20 wt %, and most preferably about 0.1 wt % to about 15 wt %, based on the total weight of the composition. When present in the foregoing amounts, especially the most preferred range, phytol synergistically enhances the effect of the retinoid, which is preferably present in the composition.

Along these lines, the compositions used in the methods of the first embodiment of the present invention may have a retinoid in an amount about 0.001 wt % to about 1.5 wt % based on the total weight of the composition. Preferably, the retinoid is present in an amount about 0.01 wt % to about 1 wt %, and most preferably about 0.1 wt % to about 0.5 wt %, based on the total weight of the composition. The amount of retinoid may be adjusted, based upon the potency of the retinoid, without departing from the present invention. Likewise, the synergistic effect is achieved within the broadest range set forth above for the phytol and the retinoid, but will vary within the ranges depending on many factors including the potency of the retinoid.

As stated above, methods according to the present invention comprise the step of topically applying one or more compositions to the skin for improving the appearance of the skin. Preferably, methods according to the present invention comprise the step of topically applying a composition as described herein at least once daily for a period of time, namely at least two days, to effect the desired improvement.

Again, methods according to the present invention may involve topically applying a single composition that has phytol and a retinoid. An alternative to the first embodiment is that two separate compositions may be contemporaneously applied, each of which has only one of either the phytol or the retinoid. The second embodiment of the methods according to the present invention comprise the step of topically applying a composition having phytol and perilla oil. For both embodiments, additional ingredients may be added as discussed below.

The improvement in the appearance of skin may be demonstrated by reducing dermatological aging, particularly dermatological aging due to intrinsic aging, such as chronological aging resulting from peri-menopausal or post menopausal changes, and extrinsic aging, such as photo-aging and effects of pollution; decreasing skin fragility; preventing and reversing loss of collagen and/or elastin; preventing skin atrophy; promoting/accelerating cell turnover; improving skin firmness/plumpness; improving skin texture; preventing and decreasing fine lines and wrinkles; improving skin tone; enhancing skin thickness; decreasing pore size; minimizing skin discoloration; restoring skin luster; minimizing signs of fatigue; improving skin barrier function; minimizing skin dryness; and preventing, reducing, or treating hyperpigmentation, especially associated with blemishes.

The second embodiment of the present invention provides methods in which the composition includes phytol, in the amounts set forth in the first embodiment, and perilla oil.

The perilla oil is present in an amount about 0.01 wt % to about 10 wt %. Preferably, the perilla oil is present in an amount about 1 wt % to about 8 wt %, and most preferably about 3 wt % to about 6 wt %. Perilla oil is the subject of pending U.S. application Ser. No. 09/521,442, filed Mar. 7, 2000, noted above, which application is incorporated herein by reference.

The compositions of the second embodiment are used in methods for improving the appearance of the human skin. In addition, these compositions of the second embodiment can also be used to prevent, reduce and/or treat acne.

Compositions useful in either embodiment of the methods of the present invention may have an acceptable topical vehicle. The vehicle must be adapted to receive, for the first embodiment, the phytol and, preferably, also the retinoid. For the second embodiment, the vehicle must be adapted to receive the phytol and the perilla oil.

Examples of the form of an acceptable vehicle are a lotion, a cream, a gel, a spray, an ointment, a serum, a patch, a foundation, a stick, and/or another form known to those skilled in the art.

The compositions useful in the methods of the first and second embodiments of the present invention may, and preferably do, include an anti-inflammatory agent. Suitable anti-inflammatory agents include, but are not limited to, salicylic acid, boswellic acid, curcumin, tetrahydrocurcumin, ferulic acid and its derivatives, rosmarinic acid, catechins, and bisabolol. Preferably, the anti-inflammatory agent is salicylic acid.

The compositions useful in the methods of the first and second embodiments of the present invention may, and preferably do, include a 5-alpha reductase inhibitor. Suitable inhibitors include, but are not limited to, saw palmetto and finasteride.

In addition, compositions useful in the methods of the first and second embodiments of the present invention may include one or more exfoliants. Suitable exfoliants include, but are not limited to, alpha-hydroxy acid, beta-hydroxy acid, keto acid, niacinamide, oxa acid or oxa diacid (disclosed in U.S. Pat. Nos. 5,847,003 and 5,834,513), and combinations thereof. Preferred alpha-hydroxy acids include lactic acid, glycolic acid or a mixture thereof. The preferred oxa diacid is 3,6,9-trioxaundecanedioic acid. Preferably, the exfoliant is or includes niacinamide.

Compositions useful in the methods of the first and second embodiments of the present invention may also include one or more hypopigmenting agents. The hypopigmenting agent includes, but is not limited to, hydroquinone ascorbic acid and/or licorice extract; and ascorbyl-phosporyl-cholesterol that is disclosed in U.S. Pat. No. 5,866,147.

Furthermore, compositions useful in the methods of the first and second embodiments of the present invention may include one or more sunscreens. Suitable sunscreens include, but are not limited to, oxybenzone, octyl salicylate, octyl methoxycinnamate, octocrylene, titanium dioxide, zinc oxide, butylmethoxydibenzoylmethane, methylene bis-benzotriazoyl tetramethylbutylphenol (MBBT), bis-ethylhexyl oxyphenol methoxyphenol triazine (BEMT), or any combinations thereof.

One or more antioxidants may be included in the compositions used in the methods of the first and second embodiments of the present invention. Suitable antioxidants include, but are not limited to, compounds having phenolic hydroxy functions, such as ascorbic acid and ascorbic acid derivatives; gallic acid and its derivatives (e.g. propyl gallate); ferulic acid and its derivatives (e.g. ethyl ferulate, sodium ferulate); nitrones; N-tertbutyl-nitrone; I-(4-pyridyl-1-oxide)-N-tertbutyl-nitrone; curcumin, tetrahydrocurcumin; 6-hydroxy-2,5,7,tetramethylchroman-2-carboxylic acid; uric acid; reductic acid; tannic acid; rosmarinic acid; tocopherol and its derivatives; catechins; and mixtures thereof. Other suitable antioxidants are those that have one or more thiol functionals (—SH), in either reduced or non-reduced form, such as glutathione, lipoic acid, thioglycolic acid, and other sulfhydryl compounds. The antioxidant may be inorganic, such as a sulfite, bisulfite, metabisulfite, or another inorganic salt and/or acid containing sulfur. Preferably, the antioxidant is or includes tetrahydrocurcumin.

Compositions useful in the methods of the first and second embodiments of the present invention may include one or more barrier function enhancing agents. Examples include ceramides; essential fatty acids and their esters, especially glycerides, α-hydroxy fatty acids and their esters, ω-hydroxy fatty acids and their esters; phospholipids; cholesterol and its esters, such as cholesteryl hemisuccinate, cholesteryl phosphate; and cholestanol and its derivatives. The barrier function enhancing agent can be added to a topical composition either as singular molecular entities or as a complex mixture of lipids derived from either synthetic, animal or plant sources.

In addition, compositions useful in the methods of the first and second embodiments of the present invention may include one or more collagen enhancing agents. These agents prevent skin sagging by promoting a net increase in collagen, either by reducing collagen breakdown or by promoting collagen formation. Examples of such agents include Clara extract (Sophora augustifolia), ascorbyl-phoshoryl-cholesterol, ascorbic acid, ascorbic acid derivatives, and mixtures thereof.

Also, compositions useful in the methods of the first and second embodiments of the present invention may include one or more elastase inhibitors. Examples of these inhibitors include fatty acids, such as oleic acid, perinaric acid, and Honeysuckle extract (Lonicera caprifolium). These inhibitors act to prevent sagging of the skin.

The compositions useful in the methods of the first and second embodiments of the present invention may include one or more phytoestrogens. Phytoestrogens are described in PCT WO 00/13661, which is incorporated herein by reference.

Compositions useful in the methods of the first and second embodiments of the present invention may include additional ingredients. Such additional ingredients may be one or more bulking agents, clays, emollients, emulsifiers, fillers, fragrances, gellants, germicides, humectants, lipid materials, moisturizers, natural oils, perfumes, polymers, preservatives, solvents, stabilizers, thickeners, ultraviolet light absorbers, skin cooling agents, skin protectants, penetration enhancers, vitamins, waxes, or any combinations thereof.

Various modifications and alterations to the present invention may be appreciated based on a review of this application. These changes and additions are intended to be within the scope and the spirit of the present invention as defined by the following claims.

What is claimed is:

1. A method of improving the appearance of human skin comprising the step of:
applying to the skin a topical composition having phytol and a retinoid, wherein said phytol is present in an amount effective to synergistically enhance the beneficial effect of said retinoid.

2. The method of claim 1, wherein said phytol is present in an amount about 0.0001 wt % to about 50 wt % based on the total weight of the composition.

3. The method of claim 1, wherein said phytol is present in an amount about 0.01 wt % to about 20 wt % based on the total weight of the composition.

4. The method of claim 1, wherein said phytol is present in an amount about 0.1 wt % to about 15 wt % based on the total weight of the composition.

5. The method of claim 1, further comprising an ingredient selected from the group consisting of an antinflammatory agent, a 5-alpha reductase inhibitor, and combinations thereof.

6. The method of claim 1, wherein the method of improving the appearance of human skin provides at least one result selected from the group consisting of: reducing dermatological aging; decreasing skin fragility; preventing and reversing loss of collagen and/or elastin; preventing skin atrophy; promoting/accelerating cell turnover; improving skin firmness/plumpness; improving skin texture; preventing and decreasing fine lines and wrinkles; improving skin tone; enhancing skin thickness; decreasing pore size; minimizing skin discoloration; restoring skin luster; minimizing signs of fatigue; improving skin barrier function; minimizing skin dryness; preventing, reducing, or treating hyperpigmentation; and any combination thereof.

7. The method of claim 1, wherein the composition further includes at least one ingredient selected form the group consisting of a vehicle, 5-alpha reductase inhibitors, an exfoliant, a hypopigmenting agent, a sunscreen, an antioxidant, barrier function enhancing agents, collagen enhancing agents, elastase inhibitors, phytoestrogen, and combinations thereof.

8. The method of claim 1, wherein the composition further includes one or more ingredients selected from the group consisting of bulking agents, clays, emollients, emulsifiers, fillers, fragrances, gellants, germicides, humectants, lipid materials, moisturizers, natural oils, perfumes, polymers, preservatives, solvents, stabilizers, thickeners, ultraviolet light absorbers, skin cooling agents, skin protectants, penetration enhancers, vitamins, waxes, or any combinations thereof.

9. The method of claim 1, wherein the composition is applied at least once a day for more than two days.

10. The method of claim 1, wherein the retinoid is retinol.

11. A method of improving the appearance of human skin comprising the step of:
applying to the skin a topical composition having phytol in an amount about 0.0001 wt % to about 50 wt % and a retinoid in an amount about 0.001 wt % to about 1.5 wt % based on the total weight of the composition, wherein said phytol is present in an amount effective to synergistically enhance the beneficial effect of said retinoid.

12. The method of claim 11, wherein said phytol is present in an amount about 0.01 wt % to about 20 wt % based on the total weight of the composition.

13. The method of claim 11, wherein said phytol is present in an amount about 0.1 wt % to about 15 wt % based on the total weight of the composition.

14. The method of claim 11, wherein the improved appearance of human skin provides at least one result selected from the group consisting of: reducing dermatological aging; decreasing skin fragility; preventing and reversing loss of collagen and/or elastin; preventing skin atrophy; promoting/accelerating cell turnover; improving skin firmness/plumpness; improving skin texture; preventing and decreasing fine lines and wrinkles; improving skin tone; enhancing skin thickness; decreasing pore size; minimizing skin discoloration; restoring skin luster; minimizing signs of fatigue; improving skin barrier function; minimizing skin dryness; and preventing, reducing, or treating hyperpigmentation, and any combination thereof.

15. A method of improving the appearance of human skin comprising the step of:
applying to the skin a topical composition having phytol and perilla oil.

16. The method of claim 15, wherein said phytol is present in an amount about 0.0001 wt % to about 50 wt % based on the total weight of the composition.

17. The method of claim 15, further comprising an ingredient selected from the group consisting of an antinflammatory agent, a 5-alpha reductase inhibitor, and combinations thereof.

18. The method of claim 17, wherein said anti-inflammatory agent is salicylic acid.

19. A composition comprising:
at least one retinoid, and phytol,
wherein said phytol is present in an amount effective to synergistically enhance the effectiveness of said retinoid.

20. The composition of claim 19, wherein said phytol is present in an amount about 0.0001 wt % to about 50 wt % based on the total weight of the composition.

21. The composition of claim 19, wherein said retinoid is present in an amount about 0.001 wt % to about 1.5 wt % based on the total weight of the composition.

22. The composition of claim 19, further comprising a component selected from the group consisting of an antinflammatory agent, 5 alpha reductase inhibitor, and combinations thereof.

23. A topical composition for improving the appearance of human skin comprising:
perilla oil, and phytol,
wherein said phytol is present in an amount effective to synergistically enhance the effectiveness of a retinoid on the skin.

24. The composition of claim 23, further comprising at least one ingredient selected from the group consisting of an antinflammatory agent, 5 alpha reductase inhibitor, and combinations thereof.

25. A method of preventing, reducing or retarding acne comprising the step of:
applying to the skin a topical composition having phytol and perilla oil.
